(19) United States
(12) Patent Application Publication
(10) Pub. No.: US 2008/0215076 A1
(43) Pub. Date: Sep. 4, 2008

(54) GASTRO-INTESTINAL THERAPEUTIC DEVICE AND METHOD

(75) Inventor: Randal S. Baker, Ada, MI (US)

Correspondence Address: VAN DYKE, GARDNER, LINN & BURKHART, LLP, SUITE 207, 2851 CHARLEVOIX DRIVE, S.E., GRAND RAPIDS, MI, 49546, US

(73) Assignee: SENTINEL GROUP, LLC, Grand Rapids, MI (US)

(21) Appl. No.: 12/116,573

(22) Filed: May 7, 2008

Related U.S. Application Data
(63) Continuation-in-part of application No. PCT/US06/60881, filed on Nov. 14, 2006.
(60) Provisional application No. 60/982,859, filed on Oct. 26, 2007.
(60) Provisional application No. 60/597,151, filed on Nov. 14, 2005.

(51) Int. Cl. A61B 17/00; A61B 17/08; A61M 29/02

(52) U.S. Cl. 606/154; 128/887; 606/153; 606/192

(57) ABSTRACT

A gastro-intestinal therapeutic device and method includes providing a therapeutic device having a body and an anchoring mechanism. The anchoring mechanism is adapted to resist distal migration of said body in a gastro-intestinal tract. The body is generally configured to a portion of the gastro-intestinal tract and has a first wall portion and a second wall portion. The first wall portion defines a generally sealed membrane. The second wall portion has a scar-forming agent.

### CROSS REFERENCE TO RELATED APPLICATIONS

The present invention claims the benefit of U.S. provisional patent application Ser. No. 60/982,859, filed on Oct. 26, 2007, and is a continuation-in-part application of International Application No. PCT/US2006/060881, filed on Nov. 14, 2006, which claims the benefit of U.S. provisional patent application Ser. No. 60/597,151, filed on Nov. 14, 2005, the disclosures of which are hereby collectively incorporated herein by reference in their entireties.

### BACKGROUND OF THE INVENTION

The present invention is directed to a therapeutic method and apparatus for the gastro-intestinal tract. While it may have other applications, the therapeutic device may be used as therapy for an anastomosis, a fistula, diverticular disease, an incision or a stricture. Also, the therapeutic device may be used in forming a stomal opening.

Anastomoses have an unacceptably high rate of leakage. This is especially the case for the anastomosis of the esophagus and bowel. Various anastomotic devices have been proposed. Such prior devices have been more concerned with the mechanical joining of the portions of the ligated luminal viscus. Moreover, prior devices often require complex procedures for joining the portions. For example, many require specialized tools to apply the device. The complexity imposed by the separate joining means and specialized tools increases the time required to make the anastomosis and limits the applications for which the prior devices may be used.

Also, prior devices are capable of being applied only at the time of the anastomosis. Should a leak develop at an anastomosis after it is completed, the prior devices are not configured to be applied at a later time. Also, prior devices are not capable of being applied to both anastomosis and fistulas.

Over 250,000 gastric bypass surgeries are performed each year in the United States. Patients typically lose up to 60 percent of their excess body weight over approximately 18 months. At least 10 percent of the patients will begin to gain weight back. The weight gain is often due to stomal dilation, or enlargement, over time. The stoma is the anastomosis site where the pouch opens to the small bowel. Such enlargement leads to the loss of satiety as the pouch empties too early. The stomal enlargement occurs irrespective of techniques used to form the stoma, such as linear staple, hand sown, EEA, or the like.

Diverticular disease, which may include an out-pouching or even a perforation of the diverticula, may require a resection of the bowel. Leaks and fistulas of the bowel are typically treated by withholding oral intake while treating the patient with various medications. Both procedures have obvious risks. The resection of the bowel is intrusive and can cause abdominal infection. Withholding oral intake is uncomfortable to the patient and risks weakening the patient. Also, some patients require parenteral feeding which further increases risks.

### SUMMARY OF THE INVENTION

A gastro-intestinal therapeutic device and method according to an aspect of the invention includes providing a therapeutic device having a body and an anchoring mechanism. The anchoring mechanism is adapted to resist distal migration of said body in a gastro-intestinal tract. The body is generally configured to a portion of the gastro-intestinal tract and has a first wall portion and a second wall portion. The first wall portion defines a generally sealed membrane. The second wall portion has a scar-forming agent.

The body may be bioabsorbable in a patient or removable. The therapeutic device is positioned at a portion of the gastro-intestinal tract and the scar-forming agent causes scar tissue to form at the portion of the gastro-intestinal tract. The body may be absorbed in the patient. The device may be used, for example, for stricture amelioration, leak and fistula control, decrease in the risk of stomal dilation and control of diverticular disease.

The therapeutic device may be positioned at a portion of the gastro-intestinal tract having at least one chosen from (i) an anastomosis, (ii) a fistula, (iii) diverticular disease, (iv) a stomal opening, (v) an incision, and (vi) a stricture. The therapeutic device may be deployed endoscopically through the esophagus or the anus or transluminally.

The therapeutic device may be used in performing transgastric surgery. The device may be positioned at an incision in the bowel formed during the transgastric surgery. The device may be positioned laparoscopically from outside the gastro-intestinal tract through an incision in the gastro-intestinal tract.

The therapeutic device may be used in creating or reinforcing a stomach pouch having a stomal site that is adapted to restrict the passage of food. The therapeutic device is positioned at said stomal site. The through-opening in the body may have a diameter that is in the range of from approximately 0.5 centimeters to approximately 1.5 centimeters. The body may be positioned at a stomal site that is formed using at least one chosen from linear stapling, hand suturing and EEA. The stomal site may be formed laparoscopically. The stomal site may be formed in a gastric bypass procedure. The body may be positioned during the same procedure as forming a stoma or after the procedure forming a stoma.

The therapeutic device may be used to repair an anastomosis post-operatively.

The anchoring mechanism may include an annular flange around said body. The anchoring mechanism may include the outer layer being of a material having tissue in-growth or tissue attachment surface characteristics. The anchoring mechanism may include anti-migration tines. The tines may be deployed in situ such as with a deployment device. The deployment device may include a balloon and the tines may be deployed by inflating the balloon. The anchoring mechanism may include mucosal capture.

Radiopaque markers may be provided on the body. The markers may be used to monitor for distal migration of the body as well as the absorption of the body in the patient.

These and other objects, advantages and features of this invention will become apparent upon review of the following specification in conjunction with the drawings.

### BRIEF DESCRIPTION OF THE DRAWINGS

FIG. 1 is a sectional view of a gastro-intestinal therapeutic device, according to an aspect of the invention;

FIG. 2 is a sectional view of a deployment device;

FIG. 3 is the same view as FIG. 1 of an alternative embodiment thereof;

FIG. 3a is the same view as FIG. 3 of an alternative embodiment thereof;

FIG. 4 is an illustration of a gastric bypass surgical procedure utilizing a gastro-intestinal therapeutic device and method according to an aspect of the invention;

FIG. 5 is a perspective view of the therapeutic device in FIG. 4;

FIG. 6 is a perspective view of a diverticular disease therapeutic device; and

FIG. 7 is the same view as FIG. 6 of an alternative embodiment thereof.

### DESCRIPTION OF THE PREFERRED EMBODIMENT

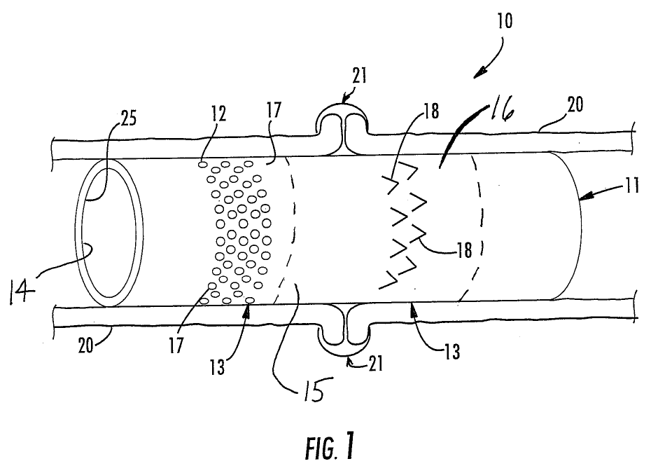

Referring now specifically to the drawings, and the illustrative embodiments depicted therein, a therapeutic device 10 is shown applied to a portion 21 of the gastro-intestinal tract 20, such as in the esophagus, colon, stomach, small intestine, large intestine, or the like (FIG. 1), to which therapy is applied. Portion 21 may be, for example, an anastomosis and may be made in a conventional fashion according to the preferences of the surgeon, such as by sutures, staples, circular stapler, linear stapler, or the like. Therapeutic device 10 includes body 11 having a wall 12 that supports portion 21 of the gastro-intestinal tract against stricture or other inward growth or collapses. This may be accomplished by making wall 12 from a material that resists inward force, such as by being made at least in part of a rigid or semi-rigid material. Alternatively, wall 12 may be made of a self-expanding material. The wall material may be bio-absorbable. Examples of suitable materials include ePTFE, silicone, or the like. Also, bio-absorbable metal-based materials are also known, as would be within the knowledge of the skilled artisan. The wall may include a plastic expandable coil in order to impart the self-expanding nature of the wall. The wall may be formed of a mesh with a sealed layer. Wall 12 may have a configuration to form an interference fit with therapeutic site 21 of the gastro-intestinal tract. The interference fit may be formed by over-sizing the diameter of wall 12 with respect to the organ, such as to form flared ends to the body and temporarily reducing the diameter of the wall to fit within the organ.

Wall 12 includes a first portion, which may be an inner layer or surface 14, middle layer or an outer layer. The first portion forms a generally sealed membrane. Inner layer 14 defines a through-opening, or lumen, 25 in the device. This facilitates passage of food and other materials, such as fecal materials, through the gastro-intestinal tract while resisting leakage of these materials to the site where therapy is occurring. Wall 12 further includes a second portion 15, which may be an outer layer or surface, which causes formation of scar tissue in the patient via a scar-forming agent 16. The scar-forming agent may be the entire extent of second portion 15, running the length of body 11, or may be discrete section(s), such as at the ends or in the middle, as shown in FIG. 1. The scar tissue provides therapeutic affects on the patient. For example, the scar tissue strengthens the wall of the gastro-intestinal tract as well as fills in fistulas, voids, and the like. Wall 12 may be a rigid or semi-rigid material. In this manner, wall 12 provides a form, or backing, that supports the portion of the site of the gastro-intestinal tract at which the device is positioned in order to resist any shrinkage resulting from the formation of scar tissue. Once the scar tissue forms, body 11 can be removed, such as by absorption in the patient, with the opening of the site maintained generally at its desired size without significant dilation or shrinkage thereof. This is because scar tissue does not readily dilate or shrink, once formed. Alternatively, body 11 can be removed through the mouth or anus.

In one embodiment, the scar-forming agent includes a sclerosant agent that is applied to the second portion 15. Alternatively, the sclerosant agent may be applied between therapeutic device 10 and the wall of the gastro-intestinal tract. Alternatively, the sclerosant agent may be incorporated into the material forming second portion 15 of sidewall 12. The sclerosant agent may be incorporated into the sidewall 12 in a manner that the absorption of the therapeutic device 10 causes release of the sclerosant agent to the site in the gastro-intestinal tract to cause the scaring. Once scar tissue has formed, sidewall 12 is no longer necessary to maintain the size of the opening in the gastro-intestinal tract. Sclerosant agents are known in the art. An example is sodium morrhuate, although any known sclerosant agent may be used.

Scar-forming agent 16 may, alternatively, be formed by the physical characteristics of second portion 15 or wall 12. For example, second portion may be formed of a copolymer glycolide and trimethylene carbonate micro porous structure, of the type that is commercially available from W.L. Gore & Associates under the SeamGuard brand. These and other structures may promote scars forming by promoting deposition of type II collagen, which is a form of scar tissue. In yet an additional alternative embodiment, scar-forming agent 16 may include an osteosynthesis material of the type available from Stryker Corporation. Such osteosynthesis material forms bone tissue locally thereby producing scarring at the site. In yet an additional alternative embodiment, scar-forming agent 16 may include tissue-ingrowth characteristics to cause fibrosis with the portion of the gastro-intestinal tract. For any of the above-described embodiments, an anti-microbial and/or anti-biotic substance, such as a silver impregnation, may be incorporated in wall 12 to produce an anti-microbial effect.

Therapeutic device 10 includes a fixation system, or anchoring mechanism, shown generally as 13 to resist distal migration of the device with respect to the gastro-intestinal tract where peristalsis tends to cause distal migration of any object in the tract. Fixation system 13 may include a surface pattern that promotes attachment of mucosal tissue to wall 12. This may include, for example, a micromesh or a macromesh in the form of a pattern of holes, or fenestrations, 17 for the purpose of promoting ingrowth of tissue through wall 12 or tissue attachment to second portion 15. The tissue attachment and/or ingrowth provides fixation from distal migration. If wall 12 is made from a bioabsorbable material, then the ingrown tissue will dissipate when the body is absorbed in the patient. Fixation system 13 may include mucosal capture fixation as disclosed in commonly assigned International Application No. PCT/US2008/053797 filed Feb. 13, 2008, for MUCOSAL CAPTURE FIXATION OF MEDICAL DEVICE, and in U.S. provisional patent application Ser. Nos. 60/901,457, filed Feb. 14, 2007; 60/921,930 filed Apr. 5, 2007; and 60/015,258 filed Dec. 20, 2007, the disclosures of which are hereby collectively incorporated herein by reference in their entireties.

Fixation system 13 may include a series of projections 18 from wall 12. Projections 18 may include barbs, V-shaped appendages, metal anchors, and the like. The projections are oriented to resist migration distally. It should be clear that more than one fixation system may be used. For example, V-shaped appendages may be utilized to temporarily fix the device while tissue is growing through the pattern of holes 17.

Other fixation techniques, such as T-shaped fasteners, staples and/or suturing may be used. T-shaped fasteners, which include a pointed barb at the end of a filament, can be projected through wall 12 into surrounding tissue and then pulled tight by retracting the filament. Staples may be applied using conventional techniques adapted to the form of a circular staple head that is capable of placing a ring of staples, or tacks, in a circumferential pattern to hold wall 12 against surrounding tissue. Such T-fasteners and/or staples may be bioabsorbable or non-absorbable.

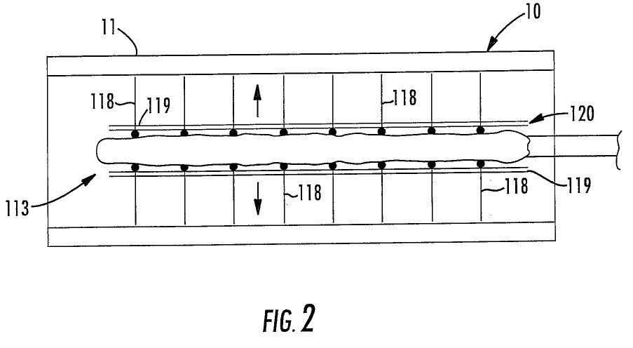

In an embodiment illustrated in FIG. 2, fixation system 113 may be balloon-deployed in situ upon placement of the therapeutic device at the site. Fixation system 113 includes a series of tines 118 that are positioned in the wall 119 of a deployment device 120. A balloon, or other expandable mechanism, inside of wall 119 is expanded, such as by inflation, in order to expand wall 119 thus thrusting the tines through the wall of body 11 and into the wall of the gastro-intestinal tract. The sealed membrane may be made from a self-sealing material, such that the tines should not create a significant leak. Alternatively, tines 118 may be formed in the wall of body 11 and projecting into opening 21 of body 11. The balloon, upon expansion, will drive the tines outwardly into the wall of the gastro-intestinal tract. The tines may be bioabsorbable or non-absorbable.

Therapeutic device 10 may be used in making an anastomosis in the gastro-intestinal tract, such as in the esophagus, bowel, or the like. In use, at the time of making anastomosis, the surgeon inserts one end of device 10 into one portion 20 of the organ and inserts the other end of device 10 into the other portion 20 of the organ at the anastomosis site. If fixation system 13 is unidirectional in operation, then care should be taken to position the device with the fixation system oriented to resist distal migration. The anastomosis is then made by the surgeon using the preferred technique of the surgeon. If it is discovered that an existing anastomosis is leaking, therapeutic device 10 may be positioned at the anastomosis site post-operatively. Dependent upon the location of the anastomosis site with respect to the organ, the therapeutic device 10 may be inserted endoscopically, by way of example, transorally, transanally, or the like. In order to accomplish such insertion through a natural orifice of the body, a conventional deployment device (not shown) may be positioned over the therapeutic device in order to compress wall 12. When the device is positioned at the anastomosis site, the deployment device is retracted from the leak protection device to deploy the leak protection device at the anastomosis site.

By providing the ability to apply therapeutic device 10 subsequent in time to performing the anastomosis, leak protection can be provided to an anastomosis that subsequently leaks. This is especially useful because it is not always possible to predict when an anastomosis may leak and, therefore, the leak protection device may not have been inserted at the time of anastomosis. Also, where the surgeon is called upon to repair a leaking anastomosis, therapeutic device 10 may be readily deployed in a minimally invasive manner.

Device 10 may also be configured with a side appendage from wall 12 to fit within a fistula to further promote scar formation. In particular, although illustrated in the context of an anastomosis, it should be apparent to the skilled artisan that therapeutic device 10 is also useful for sealing fistulas. The scar-forming agent of therapeutic device 10 forms scar tissue to close the fistula while the characteristics of wall 12 resist stricturing of the gastro-intestinal wall. Once body 11 is absorbed in the patient, or otherwise removed, the fistula is repaired. The fistula site is sealed immediately upon deployment of device 10 at the site. Device 10 is particularly useful because it can be deployed through a natural orifice of the body, such as through the mouth or the anus. Therefore, fistulas resulting from surgery, such as gastric bypass surgery, and the like, may be readily repaired when discovered in a minimally invasive manner.

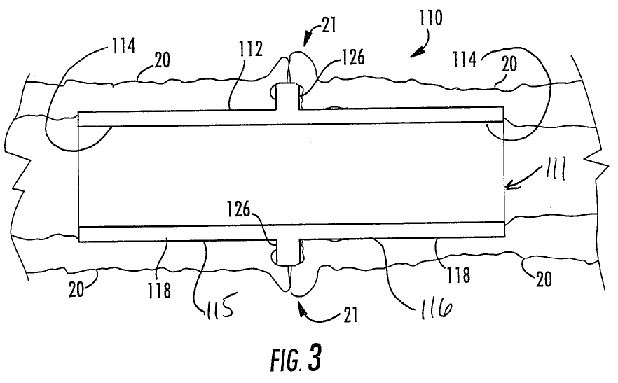

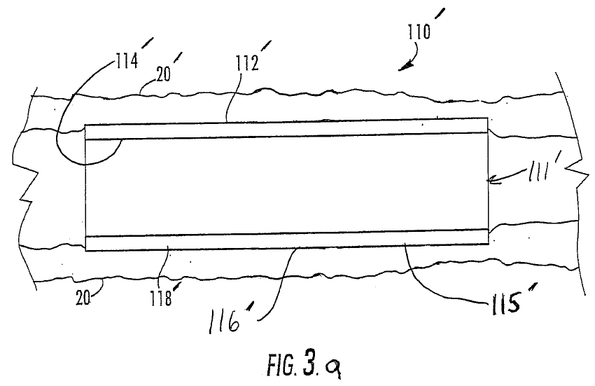

An alternative embodiment of a therapeutic device 110 includes a body 111 having wall 112 in a generally cylindrical shape (FIG. 3). Wall 112 may be rigid, semi-rigid and/or self-expanding. Wall 112 has an outer surface 115 that is configured to form an interference fit with an anastomosis site and an inner surface 114 defining a lumen for the passage of food, fecal material, or the like. Wall 112 defines a generally sealed membrane to isolate the food or fecal material from the site where therapy is occurring. Thus, device 110 may be positioned within the ends of the gastro-intestinal tract with wall 112 in a non-expanded form. This may be accomplished by a delivery mechanism (not shown) that fits over the wall and compresses the wall. After the device is properly positioned, the device is deployed from the delivery mechanism. This allows the self-expanding wall to expand into an interference fit with the lumen of the luminal viscus. Therapeutic device 110 includes a scar-forming agent 116, which may be the entire extent of outer surface 115 or only a portion thereof. Scar-forming agent 116 strengthens the wall of portions 20 of the bowel as well as fills in fistulas, voids, and the like, once device 110 is absorbed or otherwise removed.

In order to assist in providing leak protection and to resist distal migration, a flange 126 may be defined by wall 112. With the ends of the organ passing over flange 126, leakage of bowel material from the lumen will be further impeded. Also, flange 126 may provide anti-migration to prevent distal migration of device 110 within the viscus. In the embodiment illustrated in FIG. 3, body 111 has opposite end portions 118. Each end portion 118 receives and forms an interference fit with one of the gastro-intestinal tract portions 20. In addition to this interference fit and the function performed by flange 126, reinforcement may be provided to the anastomosis by suturing, stapling, or the like, used by the surgeon to join organ portions 20 together. Device 110 may be deployed at the time of making the anastomosis. Alternatively, it may be deployed later, such as when a leak occurs, by compressing wall 12 and positioning the device through a natural orifice of the body.

In another alternative embodiment illustrated in FIG. 3a, a therapeutic device 110′ includes a wall 112′ having an outer surface 115′ and an inner surface 114′ defining a lumen. Wall 112′ may be in the form of an inflatable bladder between surfaces 114′ and 115′. Wall 112′ may be inflated, using conventional techniques, in order to apply pressure with outer surface 115′ against a section 20′ of the gastro-intestinal tract. This promotes better engagement of a scar-forming agent 116′ with the gastro-intestinal tract and may serve as a fixation technique to resist distal migration.

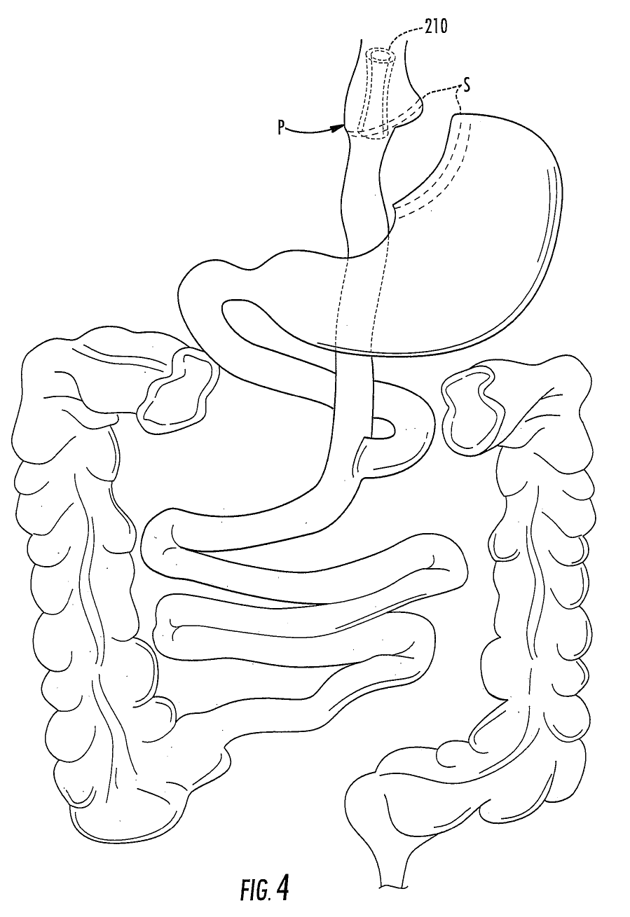

A roux-en-y gastric bypass procedure is illustrated in FIG. 4. Such procedure, which is known in the art, uses stapling shown at S to create a small, upper stomach pouch P, which restricts the amount of food which is able to be consumed. The purpose is to experience an early sense of fullness, combined with a sense of satisfaction that reduces the desire to eat. A portion of the small bowel may also be bypassed, thus delaying food from mixing with the digestive enzymes to avoid complete caloric absorption. One difficulty with known stomach pouches, or stomas, is that the opening to the jujunal of the small intestine may dilate which decreases the restrictive component of the stoma. Existing solutions, such as an outer band, may result in erosion to the conduit. Also, in certain circumstances, the opening from the stoma may experience stricture thereby requiring intervention to dilate the opening.

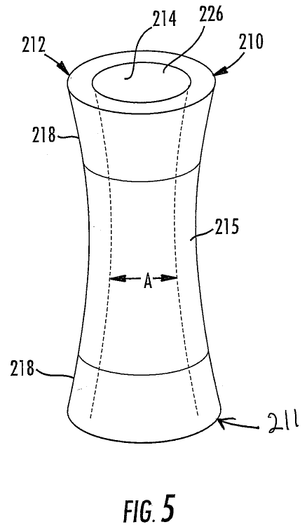

To overcome these difficulties, a therapeutic method of maintaining a stomal size includes providing a therapeutic device 210 including a body 211 having a wall 212 defining a generally sealed membrane (FIG. 5). Wall 212 includes a first wall portion 214 that defines a through-opening 226 which extends the entire length of wall 212, such that food passes through opening 226. Opening 226 is sized to have a cross-sectional area A that is selected in order to control the rate of ingested food passing through body 212. Therapeutic device 210 additionally includes an anchoring mechanism, which may include flared end portions 218 which are sized to form a tight fit with the esophagus.

Therapeutic device 210 may have an outer surface 215 which includes scar-forming agent 216, such as by a scleroscant agent, or the like, applied to an outer portion of wall 212 causes scaring of the stomal site. The scar tissue tends to contract the stoma to the shape of sidewall 212. Because sidewall portion 214 is at least semi-rigid, it resists further contraction of the stoma. Once the scar tissue forms, device 210 can be removed, such as by absorption, in the patient. The scar-forming agent may be applied directly to device 210 either by applying a coating to the outer surface 115 of wall 212 or by incorporating the sclerosant agent into the material forming wall 212 or by surface characteristics of outer surface 115 as previously described.

In the illustrative embodiment, opening 226 has a cross-sectional area A of a diameter that is sized to the patient and may range from approximately 0.5 cm to approximately 1.5 cm with approximately 1.2 cm being a nominal diameter.

Therapeutic device 210 additionally assists in preventing leaks and strictures at the jejunal junction. However, once the scar tissue is formed and any leaks healed, the stomal-sizing device can be absorbed or otherwise removed, thereby allowing the body to function without further use of an external device. While the amount of time that the therapeutic device is present in the patient may vary from patient to patient, a period of approximately six weeks may be used. Because of the scar tissue, once the therapeutic device is removed, the stoma opening should not experience significant dilation.

Other applications may be found for the therapeutic devices and methods disclosed herein. For example, they can be used to repair or preclude strictures by providing a mechanical form to keep the site open while the scar forming process more permanently retains the size of the opening. The placement of the therapeutic device at the site of the out-pouching or fistula will seal the pouched area or fistula thereby allowing food to be taken orally without causing further difficulties. The scar-forming agent will produce scar tissue to close the out-pouching or fistula. Any abscess already present can be drained such as by percutaneous drain placed with a computed axial tomography scan.

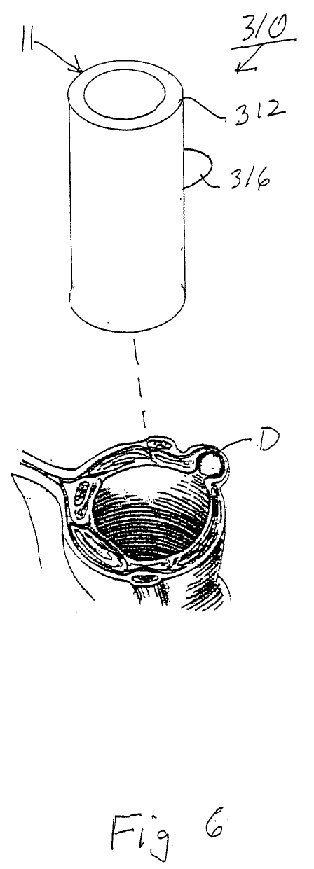

A therapeutic device may also be used with diverticular disease in the bowel. A therapeutic device 310 that is particularly useful with diverticular disease includes a body 311 having a wall 312 (FIG. 6). The wall may have an area, illustrated as an outcropping or protrusion 316 that has a configuration to generally fit within a diverticula D. Area 316 may have the general size and shape of a diverticula. The physician may position the protrusion 316 during deployment by radial and axial positioning of body 311. Radio-opaque markers and/or contrast dyes may be used such as with fluoroscopic assist for the positioning.

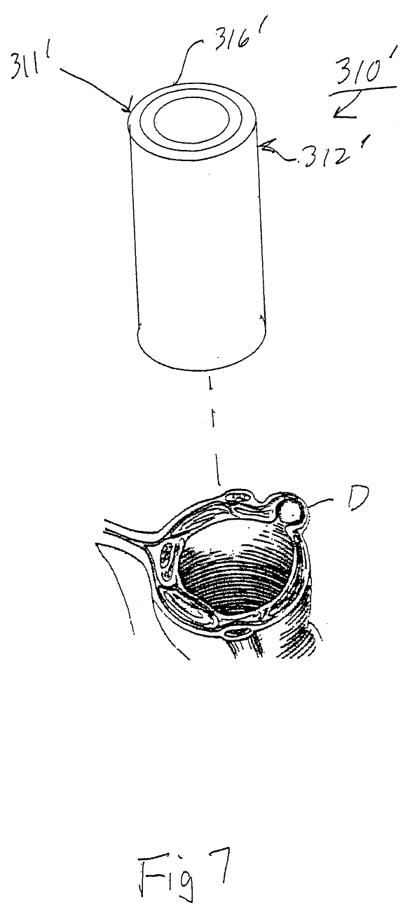

Alternatively, a therapeutic device 310′ may have a scar-forming agent in the form of a flexible cover 316′ over all or a portion of wall 312 that will generally conform to diverticula D when inserted in a patient (FIG. 7). Cover 316′ is configured to conform to minor diverticular outpouching.

Area 316, 316′ may incorporate a scar-forming agent of the type previously described. This causes scar formation in the diverticula to reduce the likelihood of perforation or stretching of the pouch and to exclude the pouch thereby isolating the pouch from the bowel. Also, area 316, 316′ is capable of applying pressure to the diverticula. This may have a tamponade effect on bleeding from vessels stretched at the outpouch with or without a scar-forming agent. This tamponade effect does not have the risks associated with cauterization of the relatively thin wall of the gastro-intestinal tract. In addition to controlling bleeding as with diverticular outpouching, devices 310, 310′ may be used to control other sources of bleeding in the gastro-intestinal tract such as bleeding associated with arteriovenous malformations (AVM).

As with prior embodiments, body 311, 311′ may be bioabsorbable in whole or in part or may be removed after scar formation and/or bleeding cessation.

The therapeutic devices and methods disclosed herein can also be used with transgastric surgery. Should the surgeon perform an incision in the bowel, either intentionally or unintentionally, a therapeutic device can be deployed using colonoscopy in order to heal the incision without formation of infection. Other applications will be apparent to the skilled artisan. Also, although illustrated as made from bioabsorbable material, in certain applications, the body of the therapeutic device can be made from a non-absorbable material and removed, such as endoscopically, upon completion of scar formation.

Also, although illustrated for application transorally or transanally, the therapeutic device can be applied otherwise, such as by transorgan or transluminal placement. Such transorgan placement may be conducted laparoscopically, using natural orifice transluminal surgery, or conventional surgery. For example, in order to repair a fistula in the bowel that is attached to the abdominal wall, making access to the site difficult, the surgeon may choose to make an incision in the bowel laparoscopically at the fistula site and make a transorgan placement of the therapeutic device.

Changes and modifications in the specifically described embodiments can be carried out without departing from the principles of the invention which is intended to be limited only by the scope of the appended claims, as interpreted according to the principles of patent law including the doctrine of equivalents.

The embodiments of the invention in which an exclusive property or privilege is claimed are defined as follows:

1. A gastro-intestinal therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism resisting distal migration of said body in a gastro-intestinal tract;
said second portion having a scar-forming agent, said scar-forming agent adapted to causing formation of scar tissue in a patient.

2. The therapeutic device as claimed in claim 1 wherein said body is bioabsorbable in a patient.

3. The therapeutic device as claimed in claim 1 wherein said scar-forming agent includes a sclerosant agent.

4. The device as claimed in claim 3 wherein said sclerosant agent comprises a coating applied to said second portion.

5. The device as claimed in claim 3 wherein said sclerosant agent is integral with said second portion.

6. The device as claimed in claim 1 wherein said scar-forming agent includes an osteosynthetic material.

7. The device as claimed in claim 1 wherein said agent comprises a physical configuration of said second portion that is adapted to causing formation of scar tissue in a patient.

8. The device as claimed in claim 7 wherein said physical configuration includes a collagen deposition region.

9. The device as claimed in claim 1 wherein said anchoring mechanism includes a mucosal capture mechanism.

10. The device as claimed in claim 1 wherein said anchoring mechanism includes an annular flange around said body.

11. The device as claimed in claim 1 wherein said anchoring mechanism includes said outer layer of a material having tissue in-growth or tissue attachment surface characteristics.

12. The device as claimed in claim 1 wherein said anchoring mechanism comprises at least one chosen from i) anti-migration tines, ii) staples and iii) T-shaped fasteners.

13. The device as claimed in claim 12 wherein said anchoring mechanism is bioabsorbable.

14. The device as claimed in claim 12 wherein said anchoring mechanism is adapted to be deployed in situ and including a deployment device, said deployment device adapted to deploying said anchoring mechanism.

15. The device as claimed in claim 14 wherein said anchoring mechanism comprises said tines and wherein said deployment device comprises a balloon, said balloon adapted to deploying said tines when inflated.

16. The device as claimed in claim 14 wherein said anchoring mechanism comprises staples and wherein said deployment device comprises a generally annular stapler.

17. The device as claimed in claim 1 wherein said anchoring mechanism comprises said wall forming an interference fit with the portion of the gastro-intestinal tract.

18. The device as claimed in claim 17 wherein said wall includes an inflatable chamber, said chamber adapted to form the interference fit with the portion of the gastro-intestinal tract when inflated.

19. The device as claimed in claim 1 wherein said second portion extends substantially the length of said wall.

20. The device as claimed in claim 1 including radiopaque markers on said body.

21. The device as claimed in claim 1 wherein said body is adapted to be deployed transorally, transanally or transluminally.

22. The device as claimed in claim 1 wherein said inner layer is rigid or semi-rigid.

23. A gastro-intestinal therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism adapted to resist distal migration of said body in a gastro-intestinal tract;
said second portion having a scar-forming agent, said scar-forming agent adapted to cause formation of scar tissue in a patient;
a through-opening defined in said body, said through-opening having a cross-sectional area that is configured to controlling the rate of ingested food passing through said body.

24. The device as claimed in claim 23 wherein said through-opening has a cross-sectional diameter that is in the range of from approximately 0.5 centimeters to approximately 1.5 centimeters.

25. The device as claimed in claim 23 wherein said body is adapted to be positioned at a stomal site.

26. A diverticular disease therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism resisting distal migration of said body in a gastro-intestinal tract;
wherein said body is configured to be positioned at a site of diverticular disease and said second portion that is adapted to generally seal a diverticula.

27. The device as claimed in claim 26 wherein said second wall portion includes a protrusion that is adapted to conform to a portion of a diverticula.

28. The device as claimed in claim 26 wherein said second wall portion includes a flexible cover.

29. The device as claimed in claim 26 wherein said second wall portion having a scar-forming agent, said scar-forming agent adapted to causing formation of scar tissue in a patient.

30. A gastro-intestinal therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism resisting distal migration of said body in a gastro-intestinal tract;
said second wall portion having a scar-forming agent, said scar-forming agent adapted to causing formation of scar tissue in a patient;
wherein said body is adapted to be positioned at a bowel incision site.

31. A gastro-intestinal therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism resisting distal migration of said body in a gastro-intestinal tract;
said second wall portion having a scar-forming agent, said scar-forming agent adapted to causing formation of scar tissue in a patient;
wherein said body is adapted to be positioned at a fistula.

32. A gastro-intestinal therapeutic device, comprising:
a body generally configured to a portion of the gastro-intestinal tract, said body having a wall including a first wall portion and a second wall portion, said first wall portion defining a generally sealed membrane;
an anchoring mechanism, said anchoring mechanism resisting distal migration of said body in a gastro-intestinal tract;
said second wall portion having a scar-forming agent, said scar-forming agent adapted to causing formation of scar tissue in a patient;
wherein said body is adapted to be positioned at a stricture.

33. A gastro-intestinal therapeutic method, comprising:
providing a therapeutic device having a body and an anchoring mechanism, said mechanism adapted to resist distal migration of said body in a gastro-intestinal tract, said body having a wall generally configured to a portion of the gastro-intestinal tract and having a first wall portion and a second wall portion, said first wall portion defining a generally sealed membrane, said second wall portion having a scar-forming agent;
positioning said therapeutic device at a portion of the gastro-intestinal tract;
causing formation of scar tissue at the portion of the gastro-intestinal tract with said scar-forming agent; and
removing said body from the patient.
